(19) United States
(12) Patent Application Publication
(10) Pub. No.: US 2024/0058524 A1
(43) Pub. Date: Feb. 22, 2024

(54) ROTARY SOLENOID MICRO ACTUATOR WITH DRIVE COIL

(71) Applicant: INSULET CORPORATION, Acton, MA (US)

(72) Inventors: Daniel ALLIS, Boxford, MA (US); Kyle BREINGAN, Lowell, MA (US)

(21) Appl. No.: 18/451,328

(22) Filed: Aug. 17, 2023

Related U.S. Application Data
(60) Provisional application No. 63/371,805, filed on Aug. 18, 2022.

(51) Int. Cl. A61M 5/142

(52) U.S. Cl. CPC A61M 5/14244; A61M 5/14216; A61M 2205/0272

(57) ABSTRACT

Disclosed are techniques, devices and systems that provide a rotary solenoid micro actuator that has a rotary solenoid core with dual magnetic drive coils and magnetic field focusing elements. One or more examples may also include a spring return and other examples may include two sets of drive coils to push and pull on at each magnetic gap. The magnetic field focusing elements are operable to increase the magnetic field. The rotary solenoid micro actuator is configured to cause a liquid drug to be expelled from a drug delivery device.

### RELATED APPLICATIONS

This application claims the benefit of U.S. Provisional Patent Application No. 63/371,805, filed Aug. 18, 2022, the entire contents of which are incorporated herein by reference in their entirety.

### BACKGROUND

For wearable medical devices, physical size is a major driver of the overall impact of therapy on the patients using them. One of the largest elements in a complex medical device (such as an insulin pump) is the pumping actuator which provides the force needed to deliver drug therapy to the patient. To maintain a small device footprint, a design objective is to optimize actuator efficiency and performance while still delivering the necessary output.

As electromechanical actuators shrink in size, the relative proportion of active elements (magnets, coils, etc.) to packaging elements decreases leading to losses in both performance and efficiency.

A cause of performance and efficiency reduction can be attributed to the reduction of total magnetic flux due to size reductions in both the permanent magnets and magnetic coils employed. Since volume is a third order relationship (i.e., L×W×H), reducing each axis by half leads to a final volume equal to ⅛ of the starting volume. However, with less magnet volume, there is less magnetic flux to induce a force between permanent magnet and coil.

In many cases the required force/torque density required of pumps is fairly high. Commercially available DC micro-motors based on existing technology have little available torque and require mechanical advantage techniques (i.e., gear reduction) which adds size, cost, and complexity to actuator solutions. Rotary motors are also designed for continual rotation at fairly high speeds, whereas pumping mechanisms tend to be reciprocal in nature meaning additional conversion is required.

While some solenoids address the issue of reciprocal motion by having a bi-polar operation (on/off, push/pull, etc.) they tend to suffer from other challenges. To produce a large output force, large magnetic fields are required. Solenoids can use the attractive force of magnetic coils on a ferromagnetic plunger, but this is limited to the field strength of the coil (current) and the magnetic permeability of the plunger. In addition, in some implementation in which a permanent magnet is added to the plunger or a ferromagnetic plunger is used, the plunger's magnetic field can interact with external electromagnetic fields which may lead to inadvertent insulin delivery if/when the drug delivery device is exposed to an external magnetic field. This additional magnet may also increase the size of the drug delivery device by increasing a key dimension, for example, the outside diameter of the reservoir including the new coil.

Unfortunately, the typical arrangement of a linear solenoid puts the plunger within the center of a coil of wire producing a magnetic field within the coil. This arrangement suffers from an increase in coil diameter to accommodate larger permanent magnet volumes. In this way, the two elements are not complimentary and lead to additional inefficiencies.

When these challenges are viewed together, it is possible to arrive at a clear problem statement for the optimization of force output, actuator size (volume), and electrical output.

It would be helpful to have a small, low-cost, micro-actuator with reciprocal motion, and maximal output force for a given electrical input.

It would also be beneficial if there were a device or algorithm that took advantage of provided data to customize insulin pump settings or algorithm parameters for each individual.

### BRIEF SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

According to an example of the disclosed subject matter, a micro actuator for a wearable drug delivery device may include a force transfer assembly, a drive coil, and a main structure. The force transfer assembly may include a first focusing element and a second focusing element, wherein the second focusing element includes a yoke. The drive coil may be operable to attract or repel the first focusing element and the second focusing element. The main structure may be configured to hold the force transfer assembly and the drive coil in alignment with one another.

In another example of a micro actuator, a micro actuator for a wearable drug delivery device is provided that includes a force transfer assembly, a first pair of drive coils, and a second pair of drive coils. The force transfer assembly includes a magnet between a first focusing element and a second focusing element. The second focusing element includes a yoke. Each drive coil of the first pair of drive coils is energized to a magnetic polarity that is opposite the other drive coil in order to attract or repel the first focusing element and the second focusing element in a first direction. Each drive coil of the second pair of drive coils is energized to a magnetic polarity that is opposite the other drive coil in order to attract or repel the first focusing element and the second focusing in a second direction opposite the first direction.

### BRIEF DESCRIPTION OF THE DRAWINGS

FIG. 1A illustrates an example of a micro actuator according to the disclosed subject matter.

FIG. 1B illustrates an exploded view of the example of the micro actuator of FIG. 1.

FIG. 2A shows a side view of a magnetic assembly of a micro actuator.

FIG. 2B illustrate examples of magnetic field lines in a fully assembled micro actuator according to an aspect of the disclosed subject matter.

FIGS. 3A and 3B show a top view of the micro actuator to illustrate an example of a theory of operation of the micro actuator shown in FIGS. 1A-2B.

FIGS. 4A and 4B show top and side views of an example of a rotary solenoid micro actuator with a single drive coil.

FIGS. 4C and 4D illustrate additional details of an example of a micro actuator that includes the rotary solenoid micro actuator with a single drive coil shown in the examples of FIGS. 4A and 4B.

FIGS. 5A-5D show another embodiment that is suitable to provide a rotary solenoid micro actuator with multiple drive coils.

FIGS. 6A-6D show a dual drive coil example similar to the example of FIGS. 4A and 4B.

FIG. 7 illustrates an example of a wearable drug delivery system that may incorporate the exemplary micro actuators described herein.

### DETAILED DESCRIPTION

The following discussion provides a detailed discussion of a micro actuator. The micro actuator examples described herein may be rotary solenoid micro actuators that include one or more drive coils.

In an example, the micro actuator 100 may include a single magnet 110 that provides a static N-S (where N is red and S is green) magnetic field and two (or a pair of) focusing elements 121 and 122. The two focusing elements 121 and 122 may be physically connected (e.g., directly) to the magnet and are configured or operable to focus the magnetic field to align with one or more drive coils 131, 132, e.g. a first drive coil and a second drive coil. In some examples, the magnet 110 and focusing elements 121,122 are configured to rotate on a spindle 140. The spindle 140 may be operable to snap fit into a main structure 115 that is configured to the drive coils 131 and 132. For example, the drive coils 131, 132 may be retained in the main structure 115 and provide controllable magnetic fields to attract or repel the static magnetic field of the magnet 110. In the example, each drive coil 131 and 132 may be individually controlled to attract or repel the static magnetic field of the magnet 110 causing rotation in either of the two rotational directions.

A yoke 150 (or similar feature) is configured to transmit the torque/force of the actuator 100 to a pump mechanism (not shown in this example). In more detail, the yoke 150 may include one or more yoke extensions 153, 155 (or yoke arms). The yoke extensions may form a yoke bearing. The yoke extensions 153, 155 may be coupled to or integrated with either the first focusing element 121 or the second focusing element 122. Alternatively, the yoke extensions 153, 155 may be coupled to or integrated with both the first focusing element 121 and the second focusing element 122. While the yoke 150 is shown in a U-shape with the yoke extensions 153 and 155 forming the extended portions of the U-shape, other shapes are envisioned such as, for example, the yoke extensions 153, 155 extending from the yoke 150 in a T-shape or a Y-shape.

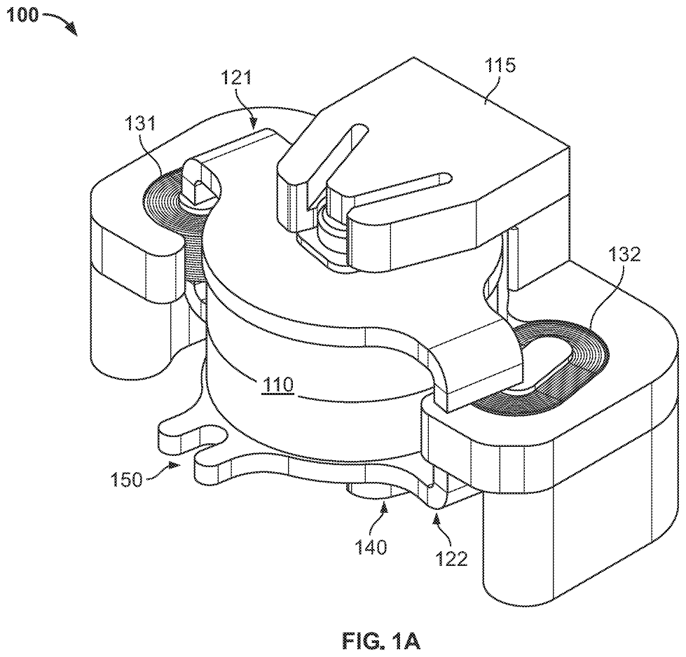

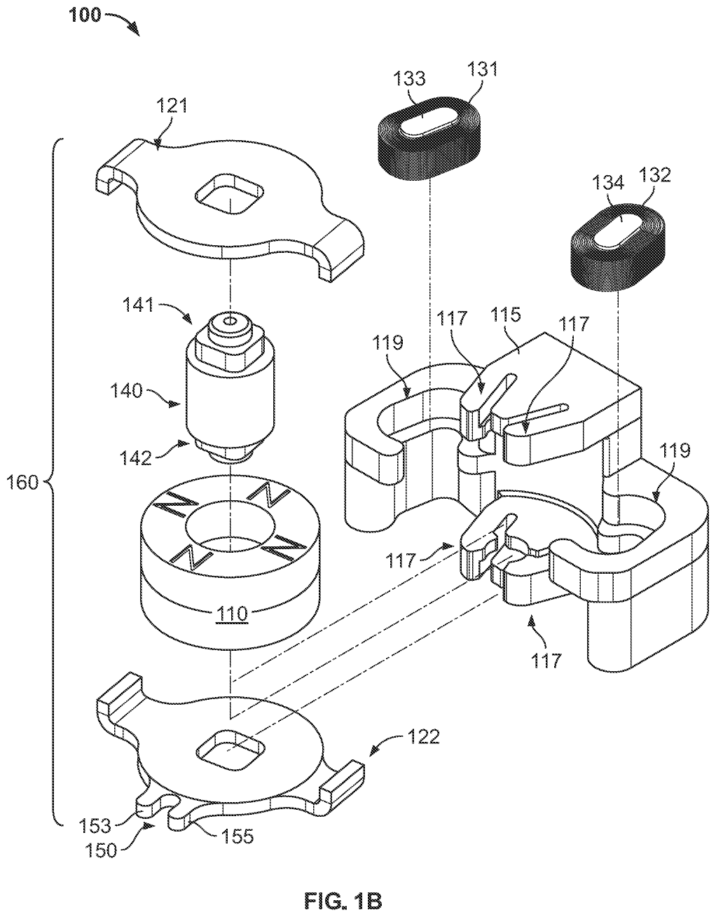

The process for assembling the structure and order of the actuator may be described with reference to FIG. 1B. The focusing elements 121 and 122 may sandwich the spindle 140 and magnet 110 to form a complete magnetic assembly 160. In some embodiments, the first focusing element includes a first spindle opening and the second focusing element includes a second spindle opening, and the spindle is configured to protrude through the first spindle opening and the second spindle opening and couple to the main structure. The first spindle opening and second spindle opening may be positioned centrally within the first and second focusing element. The square keying features 141 and 142 (also referred to as keying structure(s) or interlocking feature(s)) disposed at opposite ends of the spindle 140 ensure alignment of upper and lower focusing elements 121 and 122, respectively, to the spindle 140. A heat staking operation or the like may be used to lock the focusing elements 121, 122 and magnet 110 to the spindle 140. The complete magnetic assembly 160, once constructed, may be an inseparable assembly.

Snap features 117 or the like in a top portion and a bottom portion of the main structure 115 may be operable to allow the spindle 140 to snap into place within the main structure 115 and provide axial and radial bearing-like features for the spindle 140. The snap features 117 may be elastic. Further, the snap features 117 may each comprise a bearing surface configured to be in contact with a part of the spindle 140. In particular the snap features may each comprise a bearing surface having a circular arc shape. Accordingly, in some embodiments, the snap features are configured to receive the spindle and operable to provide axial and radial bearing-like features for the spindle. The drive coils 131 and 132 may press fit into pockets 119 of the main structure 115 to receive and locate the respective drive coils 131 and 132 in alignment with respect to the focusing elements 121, 122. The pocket 119 may function to maintain alignment and spacing of the respective drive coils 131 and 132 with or within a gap (shown in a later example) between the first focusing element 121 and the second focusing element 122. In some embodiments, the drive coil(s) are disposed within the pockets 119. In some embodiments, the first drive coil 131 and the second drive coil 132 are positioned on opposite sides of the force transfer assembly within the main structure

Ferrite cores 133 and 134 may be positioned in the respective drive coils 131 and 132 to help ensure the magnetic field lines (not shown in this example) exit the drive coils 131 and 132 in an optimized direction to interact with the permanent magnet field lines. The ferrite cores 133 and 134 in the respective drive coils 133 and 134 help improve the shape of the magnetic field to optimize the magnetic force between the magnet 110 and drive coils 131 and 132.

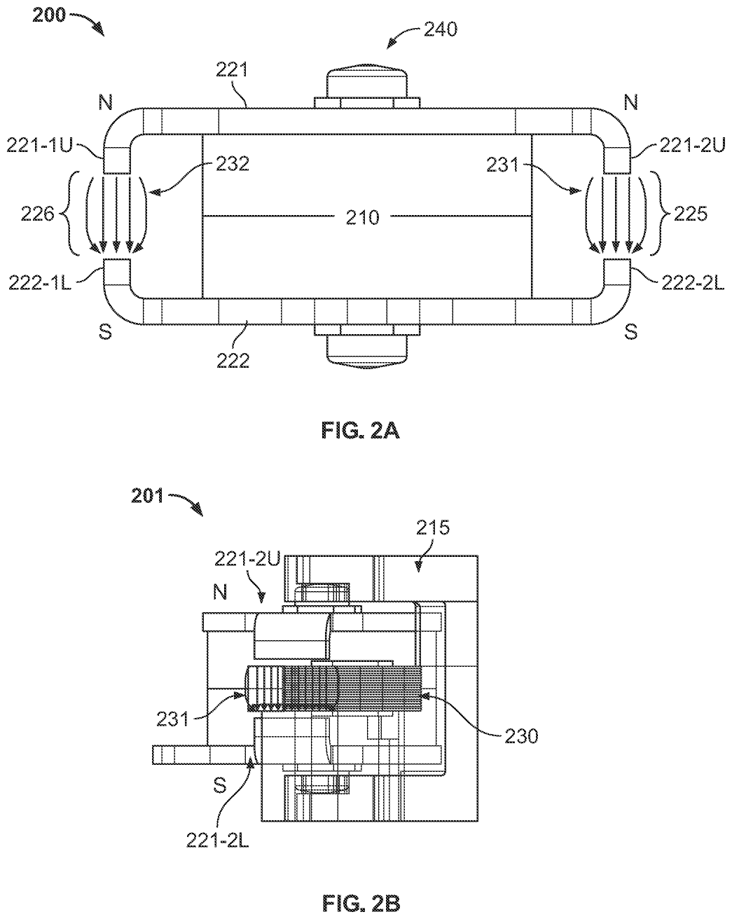

FIG. 2A shows a side view of a complete magnetic assembly of a micro actuator. FIG. 2B illustrates examples of magnetic field lines in fully assembled micro actuator according to an aspect of the disclosed subject matter.

The complete magnetic assembly 200 may include two focusing elements 221 and 222 that rotate with spindle 240 with the magnet 210 disposed between the two focusing elements 221 and 222. The focusing element 221 may be a first focusing element and focusing element 222 may be a second focusing element. Each of the focusing elements 221 and 222 of FIG. 1 may include magnetic field directing elements, e.g. elements comprising or consisting of magnetic material such as iron. The magnetic field directing elements may be distinct element or integrally formed with the focusing elements. The first focusing element may include an upper element and a lower element. In FIG. 2A, the reference number 221-1U indicates the upper magnetic field directing element (first upper magnetic field directing element) on a first side of the first focusing element 221 and the reference number 220-2U indicates another upper magnetic field directing element (second upper magnetic field directing element) on a second side of a first focusing element. The reference number 222-1L indicates the lower focusing element (first lower magnetic field directing element) on the first side of a second focusing element and 222-2L indicates the lower focusing element (second lower magnetic field directing element) on the second side of the second focusing element.

As shown on the right side of FIG. 2A, the upper magnetic field directing element 221-2U and the lower magnetic field directing element 221-2L direct the magnetic field generated by the magnet 210 (shown by lines 231) across the gap 225. In some embodiments, the upper magnetic directing element 221-2U and the lower magnetic directing element 221-2L are configured to form a gap that separates the upper magnetic directing element 221-2U from the lower magnetic directing element 221-2L. Similarly, as shown on the left side of FIG. 2A, a gap 226 is present on the opposite side of the magnet 210 and opposite to the gap 225. The magnetic field generated by the magnet 210 also crosses the gap 226 (as shown by magnetic field lines 232).

As shown in FIG. 2B, a drive coil 230 typically is positioned within the gap 225 substantially equidistant from upper focusing element 221-2U and lower focusing element 221-2L. The drive coil 230 may be held in position in a pocket of a main structure as shown in FIGS. 1A and 1B. The pocket (as shown in the earlier examples) may function to maintain alignment and spacing of the drive coil with or within the gap 225 between the first focusing element and the second focusing element. Although not shown in this example, a drive coil similar to drive coil 230 is disposed in gap 226.

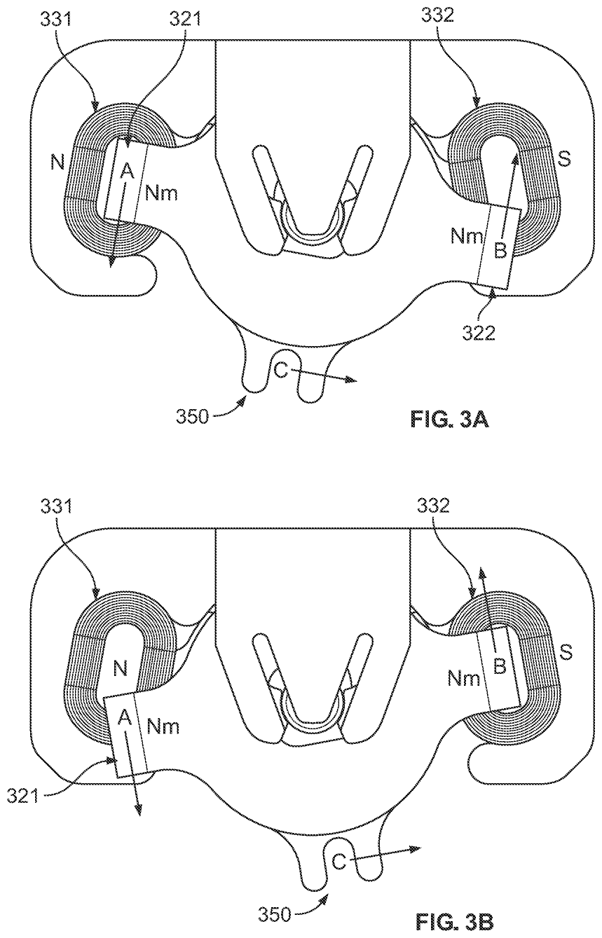

FIGS. 3A and 3B show a top view of the micro actuator to illustrate a theory of operation of the example of micro actuator as shown in FIGS. 1A through 2B.

In operation, the micro actuator 300 may be coupled to an electrical power source that is operable to energize the drive coils 331 and 332 to attract (or repel) the poles of the magnet (not shown in this example). For example, an electrical current may be applied to drive coil 331 that causes the generation of a magnetic field (in this case, N) by the drive coil 331, which may interact with a magnet, which may be a permanent magnet that emits a magnetic field Nm. The combination of the magnetic field N generated by the drive coil 331 and the magnetic field Nm emitted by the magnet produces a force that repels the focusing element 321 away from the drive coil 331 in the direction indicated by arrow A. In addition, or alternatively, an electrical current may be applied to drive coil 332 that causes the generation of a magnetic field (in this case, S) by the drive coil 332, which may interact with a magnet (e.g., a permanent magnet) that emits a magnetic field Nm. The combination of the magnetic field S generated by the drive coil 332 and the magnetic field Nm emitted by the magnet produces a force that attracts the focusing element 322 toward the drive coil 332 in the direction indicated by arrow B.

In response to the generated force that repels the focusing element 321 from the drive coil 331 and/or the generated force that attracts the focusing element 322 toward the drive coil 332, the yoke 350 translates or rotates about the spindle in the direction indicated by arrow C. Reversal of the electrical current through the drive coil(s) changes the direction of translation/rotation of the yoke 350.

Additional features may be added to the examples shown in FIGS. 1A-3B. For example, an external spring may be added to provide bi-stable positioning of the actuator at the end of a stroke. Accordingly, only one direction of current need be applied since a spring can cause the yoke 350 to rotate back to its original position (i.e., the position before a current was applied). Similarly, detents can be added to the spindle to also provide bi-stable positioning of the actuator at the end of stroke. Moreover, both an external spring and detents in the spindle may be added to provide even further bi-stable positioning of the actuator at the end of stroke beyond the single use of either the external spring or the detents in the spindle.

In a control example, the drive coils may be energized simultaneously to provide the repulsive and attractive magnetic forces. Alternatively, the drive coils may be energized in a staggered or sequential order (e.g., the attractive drive coil may be energized first, and the repulsive drive coil may be energized second) which may improve electrical efficiency.

In a further example, when the drive coils are energized, energy may be stored in the drive coils for a period of time and may be harvested utilizing a capacitive circuit or the like. In an example, the drive coil may be treated as an inductor which stores energy and only discharges stored energy after it stops receiving a current input. This stored energy can either be used to shorten the duration that the drive coil is energized or be transferred into a capacitor or the like to be reused at a later time.

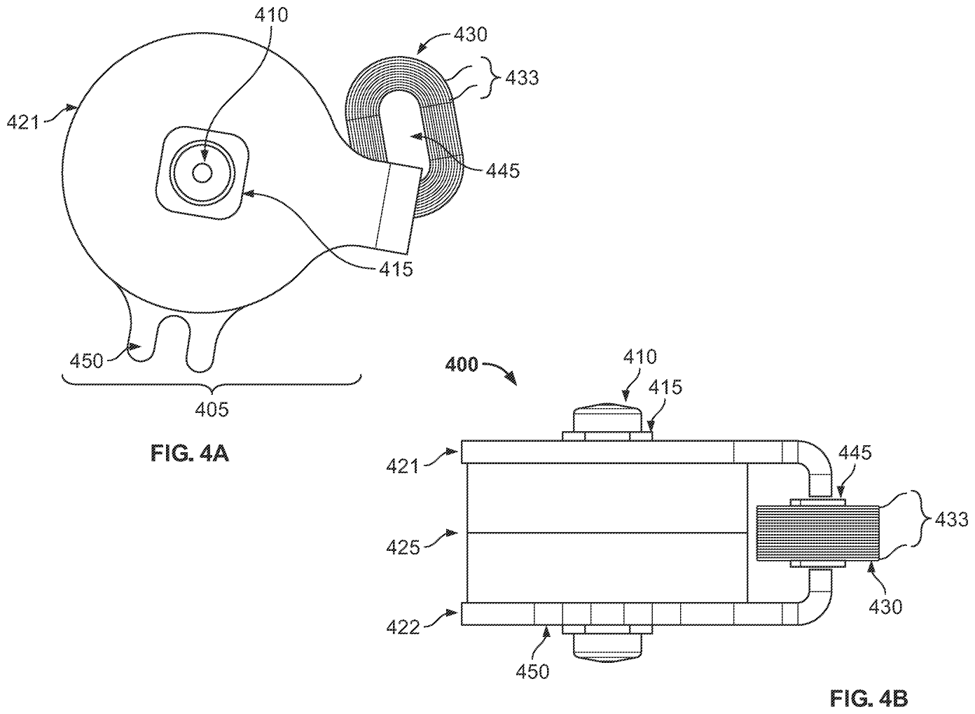

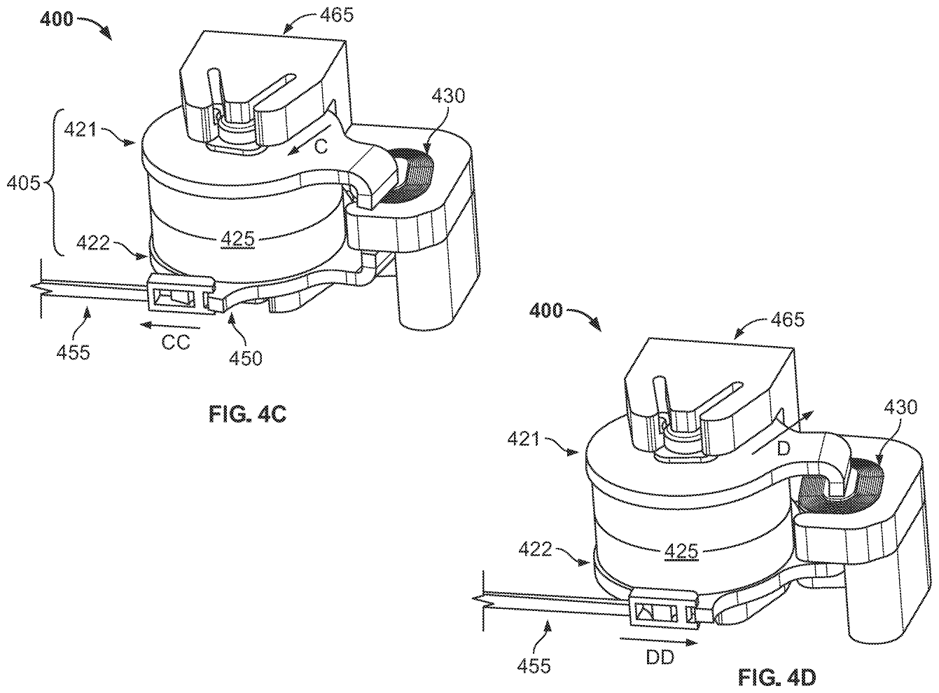

FIGS. 4A and 4B show top and side views of an example of force transfer assembly with a single drive coil that is part of a single drive coil rotary solenoid micro actuator.

A top view of the force transfer assembly 405 and single drive coil 430 are shown in FIG. 4A. This top view of the force transfer assembly 405 and single drive coil 430 shows a spindle 410, a keying feature 415 (also referred to keying structure or interlocking feature), a top focusing element 421, the single drive coil 430 with a ferrite core 445. The drive coil 430 may be energized via electrical connections 433. As shown in a later example, the electrical connections 433 may be coupled to control circuitry. Also shown in FIG. 4A is a yoke 450 of a bottom focusing element that is hidden from view beneath the top focusing element 421. The top focusing element 421 hides the magnet in FIG. 4A. The force transfer assembly and single drive coil 400 differ from earlier examples by using a single gap and drive coil instead of splitting the magnetic field focusing between two drive coils.

The side view of the force transfer assembly 405 and single drive coil 430 shown in FIG. 4B includes additional details such as the magnet 425 disposed between the top focusing element 421 and the bottom focusing element 422. The side view also shows the ferrite core 445 extending through the single drive coil 430. In this example, the ferrite core 445 extends above a top edge and below a bottom edge of the single drive coil 430. The top focusing element 421 is configured in a manner similar to the focusing element 221 with an upper focusing element curving downward toward the drive coil 430, and the bottom focusing element 422 being configured similar to the focusing element 222 with a lower focusing element curving upward toward the drive coil 430.

FIGS. 4C and 4D illustrate additional details of an example of a micro actuator that includes the rotary solenoid micro actuator with a single drive coil shown in the examples of FIGS. 4A and 4B. FIGS. 4C and 4D illustrate an example of a micro actuator 400 that includes a main structure 465 for supporting the force transfer assembly 405 and the single drive coil 430. The drive coil 430 may be held within a pocket to ensure alignment of the drive coil 430 with the focusing elements 421 and 422.

In an operational example of the micro actuator 400 described with reference to FIG. 4C, the drive coil 430 may be energized to have a magnetic polarity that repels the magnetic field created by magnet 425 and directed toward the drive coil 430 by the focusing elements 421 and 422. In response to the energizing of the drive coil 430, the force transfer assembly 405 may rotate in the direction indicated by Arrow C. An example of a drive mechanism coupling 455 is shown in FIGS. 4C and 4D. The drive mechanism coupling 455, in particular a coupling element thereof, may be in contact with the yoke. In particular, the drive mechanism coupling 455, in particular a coupling element thereof, may be disposed between the yoke extensions. The drive mechanism coupling 455 is actuated by the yoke 450 in the focusing element 422 and responds to the rotation (in the direction of Arrow C) of the force transfer assembly 405 by moving in the direction indicated by Arrow CC. The motion of the drive mechanism coupling 455 in the direction indicated by Arrow CC may cause a pump to initiate transfer of a liquid drug or the like from a reservoir by using the reciprocating motion of the drive mechanism. In some embodiments, the yoke 450 actuates a drive mechanism to expel a first amount of a liquid drug from a wearable drug delivery device. In some embodiments, movement of the yoke 550 in a first direction actuates a drive mechanism to expel a first amount of a liquid drug from a wearable drug delivery device and movement of the yoke in a second direction actuates the drive mechanism to expel a second amount of a liquid drug from a wearable drug delivery device. The rotation of the force transfer assembly 405 in the direction of Arrow CC may be stopped in a variety of ways, such as by de-energizing drive coil 430, energizing the drive coil 430 to produce an opposite magnetic polarity (i.e., the magnetic field attracts the focusing elements 421 and 422), by a mechanical stop, or the like. As shown in FIG. 4D, the drive coil 430 may be energized to have a magnetic polarity that attracts the magnetic field created by magnet 425 and directed toward the drive coil 430 by the focusing elements 421 and 422. In response to the energizing of the drive coil 430, the force transfer assembly 405 may rotate in the direction indicated by Arrow D. The drive mechanism coupling 455 is actuated by the yoke 450 in the focusing element 422 and responds to the rotation (in the direction of Arrow D) of the force transfer assembly 405 by moving in the direction indicated by Arrow DD. The motion of the drive mechanism coupling 455 in the direction indicated by Arrow DD may cause the pump to enter another stage (or the like) of the transfer of the liquid drug or the like from the reservoir.

The micro actuator 400 may also include a bias spring (not shown in this example) to ensure the yoke 450 returns to a starting position after each actuation of the yoke 450. The biasing spring may allow for only using one direction of current applied to drive coil 430, such that the magnetic field moves the yoke in one direction and the biasing spring moves the yoke in the opposite direction.

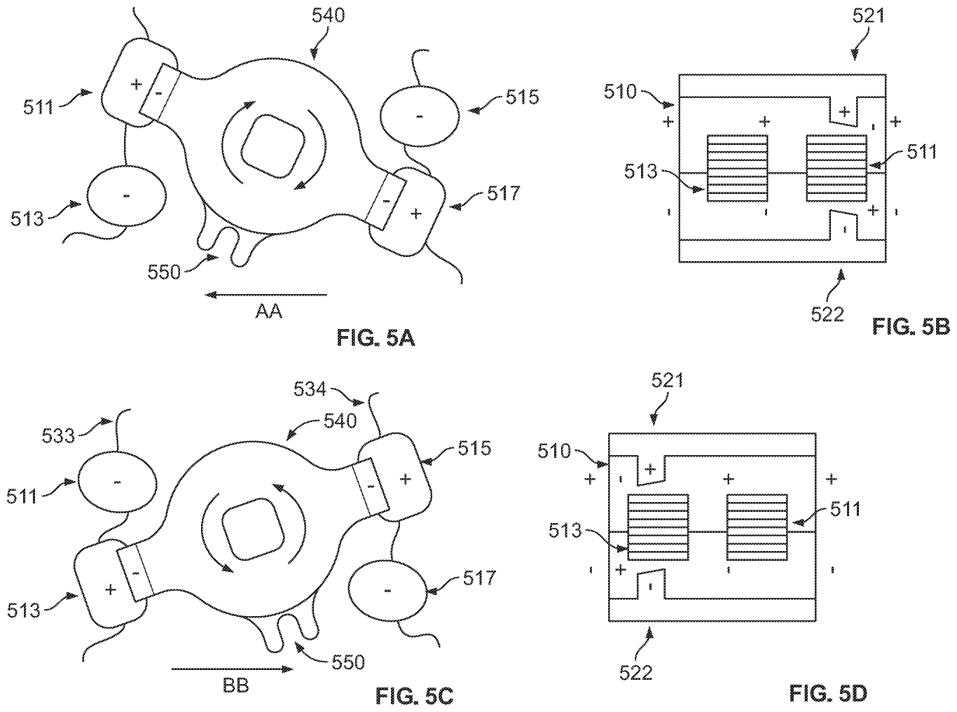

FIGS. 5A-5D show another embodiment that is suitable to provide a quadruple drive coil rotary solenoid micro actuator. FIG. 5A shows a sketch of a force transfer assembly 540 configured to operate with four drive coils 511, 513, 515 and 517. For example, the oval drive coils in the earlier examples, such as 131 and 132 of FIG. 1A, may be split into two oppositely wound coils 511/513 and 515/517 on respective sides of the force transfer assembly 540. The two oppositely wound coils 511/513 may be referred to as a first pair of drive coils and the two oppositely wound coils 515/517 may be referred to as a second pair of drive coils. The two oppositely wound drive coils 511/513 and 515/517 on each side may provide additional initial driving force (as compared to the single drive coils 131 and 132 of FIG. 1A) and help position the yoke 550. The force transfer assembly 540 and four drive coils 511, 513, 515 and 517 may be coupled to control circuitry and a drive mechanism of a wearable drug delivery system (both shown in a later example). In some embodiments, the main structure further comprises a pocket to receive and hold each respective drive coil of the first pair of drive coils and each respective drive coil of the second pair of drive coils, and wherein the pocket is configured to maintain alignment of each respective drive coil of the first pair of drive coils and each respective drive coil of the second pair of drive coils with a gap between the first focusing element and the second focusing element. In some embodiments, the main structure further comprises a plurality of pocket each comprising a respective drive coil of the first pair of drive coils and of the second pair of drive coils, and wherein the pocket is configured to maintain alignment of each respective drive coil of the first pair of drive coils and each respective drive coil of the second pair of drive coils with a gap between the first focusing element and the second focusing element.

FIG. 5B provides a sketch showing the positioning of the focusing elements 521 and 522 with respect to drive coil 511 that has been energized to attract the focusing elements 521 and 522. The two oppositely wound drive coils on each side (e.g., 511 and 513, 515 and 517) of the force transfer assembly 540 may be wound from the same wire (a first coil wound in a first direction and the second coil wound in a second direction opposite the first direction of the wire wound around the first coil), such as electrical connection 533 and electrical connection 534, to simplify the manufacturing process. In an operational example explained with reference to both FIGS. 5A and 5B, a control circuit (shown in another example) may energize drive coils 513 and 515 to cause the drive coils 511 and 517 to have a magnetic polarity that attracts the focusing elements 521 and 522, while drive coils 513 and 515 are energized to have a magnetic polarity that repels the focusing elements 521 and 522. The magnet 510 assists with the attractive forces generated by the respective drive coils 511 and 517 and with the repelling forces generated by the respective drive coils 513 and 515. In this first state, the yoke 550 is moved in the direction of Arrow AA. FIGS. 5A and 5B show the yoke 550 after it has moved in the direction of Arrow AA. The yoke 550 may interact with a drive mechanism that causes a first amount of a liquid drug to be expelled from a wearable drug delivery device.

Conversely, as shown in FIGS. 5C and 5D, the control circuit (shown in another example) may energize drive coils 513 and 515 to cause the drive coils 515 and 513 to generate a magnetic polarity that attracts the focusing elements 521 and 522, while drive coils 511 and 517 are energized to generate a magnetic polarity that repels the focusing elements 521 and 522. The magnet 510 assists with the attractive forces generated by the respective drive coils 513 and 515 and with the repelling forces generated by the respective drive coils 511 and 517. In this second state, the yoke 550 is moved in the direction of Arrow BB. FIGS. 5C and 5D show the yoke 550 after it has moved in the direction of Arrow BB. The movement of the yoke 550 in the direction of Arrow BB may also cause a drive mechanism of a wearable drug delivery device to cause a second amount of the liquid drug to be expelled from a reservoir of the wearable drug delivery device. In some embodiments, micro actuator further comprises a spindle, wherein the magnet has an opening through which the spindle passes, the first focusing element includes a first spindle opening and the second focusing element includes a second spindle opening, and the spindle is configured to protrude through the first spindle opening and the second spindle opening, in particular wherein the spindle includes at least one, in particular two, keying structure(s) configured to interlock with the first spindle opening and/or the second spindle opening

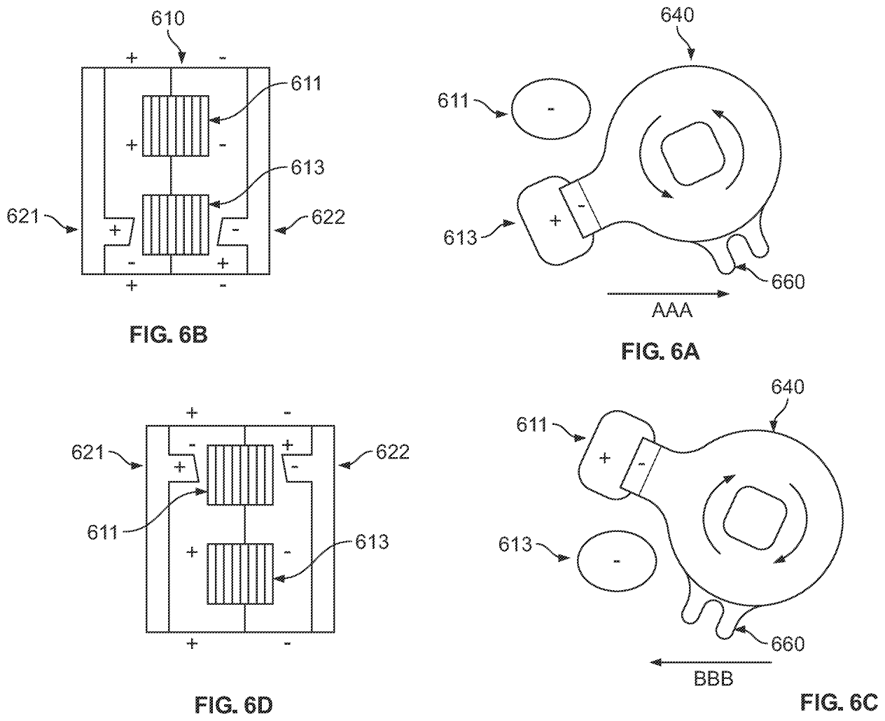

FIGS. 6A-6D show a dual drive coil example similar to the example of FIGS. 4A and 4B.

In this example as shown in FIG. 6A (on the right), one pair of oppositely wound drive coils 611 and 613 on one side of the force transfer assembly 640 may provide the directional control and positional setup of a yoke 660 required if the output torque generated by the drive coils 611 and 613 and magnet 610 (shown in FIG. 6B) is sufficient to drive a drive mechanism of a wearable drug delivery device. The two oppositely wound drive coils 611 and 613 may provide additional initial driving force and help position the yoke 660. The force transfer assembly 640 and two drive coils 611 and 613 may be coupled to control circuitry and a drive mechanism of a wearable drug delivery system.

The two oppositely wound drive coils (e.g., 611 and 613) of the force transfer assembly 640 may be wound from the same wire (a first coil wound in a first direction and the second coil wound in a second direction opposite the first) to simplify the manufacturing process. FIG. 6B provides a sketch showing the positioning of the focusing elements 621 and 622 with respect to drive coil 613 that has been energized to attract the focusing elements 621 and 622 as well as magnet 610. In an operational example explained with reference to both FIGS. 6A and 6B, a control circuit (shown in another example) may energize drive coil 613 to cause the drive coil 613 to attract the focusing elements 621 and 622, while drive coil 611 may be energized to repel the focusing elements 621 and 622. The magnet 610 may assist with the attractive forces generated by the drive coil 613 and with the repelling forces generated by the respective drive coil 611. In this first state, the yoke 660 is moved in the direction of Arrow AAA. FIGS. 6A and 6B show the yoke 660 after it has moved in the direction of Arrow AAA. The yoke 660 may interact with a drive mechanism that causes a first amount of a liquid drug to be expelled from a wearable drug delivery device.

Conversely, as shown in FIGS. 6C and 6D, the control circuit (shown in another example) may energize drive coil 611 to cause the drive coil 611 to attract the focusing elements 621 and 622, while drive coil 613 may be energized to repel the focusing elements 621 and 622. The magnet 610 may assist with the attractive forces generated by the drive coil 611 and with the repelling forces generated by the respective drive coil 513. In this second state, the yoke 660 is moved in the direction of Arrow BBB. FIGS. 6C and 6D show the yoke 660 after it has moved in the direction of Arrow BBB. The movement of the yoke 660 in the direction of Arrow BBB may also cause the drive mechanism to cause a second amount of the liquid drug to be expelled from the wearable drug delivery device.

The force transfer assembly 640 and drive coils 611 and 613 may be held in a structure similar to the main structure 115 of FIGS. 1A and 1B. The micro actuators described herein may be used in conjunction with a computer controls, e.g. medication delivery algorithms.

A type of medication delivery algorithm (MDA) may include an “artificial pancreas” algorithm-based system, or more generally, an artificial pancreas (AP) application. For ease of discussion, the computer programs and computer applications that implement the medication delivery algorithms or applications may be referred to herein as an “AP application.” An AP application may be configured to provide automatic delivery of insulin based on an analyte sensor input, such as signals received from an analyte sensor, such as a continuous blood glucose monitor, ketone sensor, or the like. The signals from the analyte sensor may contain blood glucose measurement values, timestamps, or the like.

In addition, or alternatively, while the disclosed examples may have been described with reference to a closed loop algorithmic implementation, variations of the disclosed examples may be implemented to enable open loop use. The open loop implementations allow for use of different modalities of delivery of insulin such as smart pen, syringe or the like. For example, the disclosed AP application and algorithms may be operable to perform various functions related to open loop operations, such as the generation of prompts requesting the input of information such as diabetes type, weight or age. Similarly, a dosage amount of insulin may be received by the AP application or algorithm from a user via a user interface. Other open-loop actions may also be implemented by adjusting user settings or the like in an AP application or algorithm.

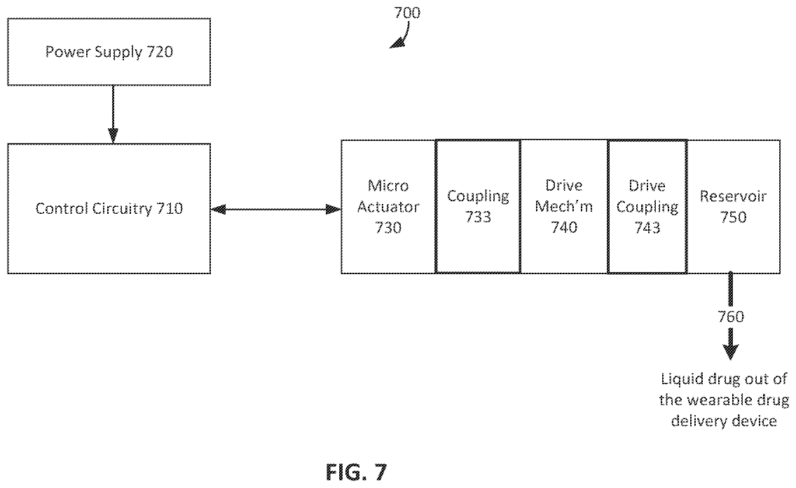

FIG. 7 illustrates an example of a wearable drug delivery system that may incorporate the example micro actuators described herein.

The wearable drug delivery system 700 may include control circuitry 710, a power supply 720, a micro actuator 730, a drive mechanism 740 and a reservoir 750. The wearable drug delivery device 700 may be a wearable device that is worn on the body of the user. The wearable drug delivery device 700 may be directly coupled to a user (e.g., directly attached to a body part and/or skin of the user via an adhesive, or the like). In an example, a surface of the wearable drug delivery device 700 may include an adhesive to facilitate attachment to the skin of a user.

The micro actuator 730 may be similar to the micro actuators with the force transfer assembly and drive coil configurations shown in the earlier examples of FIGS. 1A-6D. The reservoir 750 may store a liquid drug. Examples of a liquid drug may be or include any drug in liquid form capable of being administered by a drug delivery device via a subcutaneous cannula, including, for example, insulin, glucagon-like peptide-1 (GLP-1), pramlintide, glucagon, co-formulations of two or more of GLP-1, pramlintide, and insulin; as well as pain relief drugs, such as opioids or narcotics (e.g., morphine, or the like), methadone, arthritis drugs, hormones, such as estrogen and testosterone, blood pressure medicines, chemotherapy drugs, fertility drugs, or the like.

The micro actuator 730 may be physically coupled to the drive mechanism 740 via a coupling 733. The coupling 733 may be a mechanical structure separate from the drive mechanism 740 that couples the yoke (shown in other examples) of the micro actuator 730 to the drive mechanism 740 or may be a mechanical structure that is an integrated part of the drive mechanism 740. The coupling 733 may extend the motion of the yoke of the micro actuator to the drive mechanism or may translate the motion of the yoke to motion in a different plane or from linear motion to rotary motion or the like. Alternatively, the coupling 733 may be omitted and the yoke of the micro actuator 730 may couple directly to the drive mechanism 740.

The drive mechanism 740 may include a number of mechanical elements such as gears, gear trains and the like, that convert the linear motion of the yoke into motion in another form, such as a rotary motion of the like. The drive mechanism 740 may also include an elongated shaft that couples to a first interface (i.e., a plunger) or another interface of the reservoir 750.

A drive coupling 743 may couple the drive mechanism 740 to the reservoir 750. The drive coupling 743 may have the structural elements that enable an amount of a liquid drug stored in the reservoir 750 to be expelled from the reservoir 750 via a fluid pathway 760.

The power supply 720 may be an electrical power source, such as a battery, a super capacitor, an energy harvesting circuit or the like. The power supply 720 may be configured to last several hours, several days or the like. The power supply 720 may be replaceable and/or rechargeable via wired connections or wireless connections.

The wearable drug delivery device 700 may include control circuitry 710 that may be implemented in hardware, software, or any combination thereof. The control circuitry 710 may, for example, be a microprocessor, a logic circuit, a field programmable gate array (FPGA), an application specific integrated circuit (ASIC) or a microprocessor coupled to a memory. The control circuitry 710 may be operable to perform a number of functions, such as executing a control application stored in the memory (not shown). In the present examples, the control circuitry 710 is operable to execute logic that causes a control signal in the form of a voltage or a current to be applied to the micro actuator 730 to energize via electrical connections (shown in earlier examples) one or more drive coils as described with reference to the examples of FIGS. 1A-6D.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, novel subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible considering this disclosure. It is intended that the scope of the present disclosure be limited not by this detailed description, but rather by the claims appended hereto. Future filed applications claiming priority to this application may claim the disclosed subject matter in a different manner and may generally include any set of one or more features as variously disclosed or otherwise demonstrated herein.

Although the present invention is defined in the attached claims, it should be understood that the present invention can also (alternatively) be defined in accordance with the following embodiments:
1. A micro actuator for a wearable drug delivery device, comprising:
   a force transfer assembly including a first focusing element and a second focusing element, wherein the second focusing element includes a yoke;
   a drive coil, wherein the drive coil is operable to attract or repel the first focusing element and the second focusing element; and
   a main structure configured to hold the force transfer assembly and the drive coil in alignment with one another.
2. The micro actuator of claim 1, further comprising:
   a spindle, wherein the first focusing element includes a first spindle opening and the second focusing element includes a second spindle opening, and the spindle is configured to protrude through the first spindle opening and the second spindle opening and couple to the main structure.
3. The micro actuator of claim 1, further comprising:
   a magnet disposed between the first focusing element and the second focusing element.
4. The micro actuator of claim 1, wherein the main structure further comprises:
   snap features configured to receive the spindle and operable to provide axial and radial bearing-like features for the spindle.
5. The micro actuator of claim 1, wherein the main structure further comprises:
   a pocket to receive and hold the drive coil.
6. The micro actuator of claim 1, wherein the pocket is further operable to:
   maintain alignment of the drive coil within a gap between the first focusing element and the second focusing element.
7. The micro actuator of claim 1, wherein the drive coil comprises:
   a first drive coil and a second drive coil,
   wherein the first drive coil and the second drive coil are positioned on opposite sides of the force transfer assembly within the main structure.
8. The micro actuator of claim 1, wherein the first focusing element comprises:
   a first upper magnetic directing element and a second upper magnetic directing element disposed across from one another on opposite sides of the first focusing element.
9. The micro actuator of claim 1, wherein:
   the first focusing element includes:
   an upper magnetic field directing element disposed on a side of the first focusing element; and
   the second focusing element includes:
   a lower magnetic directing element disposed on a side of the second focusing element beneath the upper magnetic field directing element,
   wherein the upper magnetic directing element and the lower magnetic directing element are configured to form a gap that separates the upper magnetic directing element from the lower magnetic directing element.
10. The micro actuator of claim 1, wherein the yoke comprises:
   yoke extensions operable to interact with a drive mechanism of the wearable drug delivery device.
11. The micro actuator of claim 1, wherein the yoke comprises:
   yoke extensions extending from either the first focusing element or the second focusing element, wherein the yoke extensions cause the yoke to have a T-shape, a U-shape, or a Y-shape.
12. A micro actuator for a wearable drug delivery device, comprising:
   a force transfer assembly including a magnet between a first focusing element and a second focusing element, wherein the second focusing element includes a yoke;
   a first pair of drive coils; and
   a second pair of drive coils,
   wherein each drive coil of the first pair of drive coils is energized to a magnetic polarity that is opposite the other drive coil in order to attract or repel the first focusing element and the second focusing element in a first direction, and
   each drive coil of the second pair of drive coils is energized to a magnetic polarity that is opposite the other drive coil in order to attract or repel the first focusing element and the second focusing in a second direction opposite the first direction.
13. A micro actuator of claim 12, comprising:
   a main structure configured to hold the force transfer assembly, each drive coil of the first pair of drive coils, and each drive coil of the second pair of drive coils in alignment to enable rotation of the force transfer element.
14. The micro actuator of claim 13, wherein the main structure further comprises:
   snap features configured to receive the spindle and operable to provide axial and radial bearing-like features for the spindle.
15. The micro actuator of claim 13, wherein the main structure further comprises:
   a pocket to receive and hold each respective drive coil of the first pair of drive coils and each respective drive coil of the second pair of drive coils, and
   wherein the pocket is configured to maintain alignment of each respective drive coil of the first pair of drive coils and each respective drive coil of the second pair of drive coils with a gap between the first focusing element and the second focusing element.
16. The micro actuator of claim 12, wherein each drive coil of the first pair of drive coils is wound from the same piece of wire, wherein the wire is wound in a first direction for a first respective drive coil of the first pair of drive coils and in a second and opposite direction for a second respective drive coil of the first pair of drive coils.
17. The micro actuator of claim 12, wherein the yoke comprises:
   yoke extensions operable to interact with a drive mechanism of the wearable drug delivery device.
18. The micro actuator of claim 12, wherein the yoke comprises:
   yoke extensions extending from either the first focusing element or the second focusing element in which the yoke has a T-shape, a U-shape, or a Y-shape.
19. The micro actuator of claim 12, further comprising:
   a spindle,
   wherein:
   the magnet has an opening through which the spindle passes,
   the first focusing element includes a first spindle opening and the second focusing element includes a second spindle opening, and
   the spindle is configured to protrude through the first spindle opening and the second spindle opening.
20. The micro actuator of claim 19, wherein:
   the spindle includes a keying structure configured to interlock with the first spindle opening and the second spindle opening.

What is claimed is:

1. A micro actuator for a wearable drug delivery device, comprising:
a force transfer assembly including a first focusing element and a second focusing element, wherein the second focusing element includes a yoke;
a drive coil, wherein the drive coil is operable to attract or repel the first focusing element and the second focusing element; and
a main structure configured to hold the force transfer assembly and the drive coil in alignment with one another.

2. The micro actuator of claim 1, further comprising:
a spindle, wherein the first focusing element includes a first spindle opening and the second focusing element includes a second spindle opening, and the spindle is configured to protrude through the first spindle opening and the second spindle opening and couple to the main structure.

3. The micro actuator of claim 1, further comprising:
a magnet disposed between the first focusing element and the second focusing element.

4. The micro actuator of claim 1, wherein the main structure further comprises:
snap features configured to receive the spindle and operable to provide axial and radial bearing-like features for the spindle.

5. The micro actuator of claim 1, wherein the main structure further comprises:
a pocket to receive and hold the drive coil.

6. The micro actuator of claim 1, wherein the pocket is further operable to:
maintain alignment of the drive coil within a gap between the first focusing element and the second focusing element.

7. The micro actuator of claim 1, wherein the drive coil comprises:
a first drive coil and a second drive coil,
wherein the first drive coil and the second drive coil are positioned on opposite sides of the force transfer assembly within the main structure.
wire wound about the perimeter of a ferrite core.

8. The micro actuator of claim 1, wherein the first focusing element comprises:
a first upper magnetic directing element and a second upper magnetic directing element disposed across from one another on opposite sides of the first focusing element.

9. The micro actuator of claim 1, wherein:
the first focusing element includes:
an upper magnetic field directing element disposed on a side of the first focusing element; and
the second focusing element includes:
a lower magnetic directing element disposed on a side of the second focusing element beneath the upper magnetic field directing element,
wherein the upper magnetic directing element and the lower magnetic directing element are configured to form a gap that separates the upper magnetic directing element from the lower magnetic directing element.

10. The micro actuator of claim 1, wherein the yoke comprises:
yoke extensions operable to interact with a drive mechanism of the wearable drug delivery device.

11. The micro actuator of claim 1, wherein the yoke comprises:
yoke extensions extending from either the first focusing element or the second focusing element, wherein the yoke extensions cause the yoke to have a T-shape, a U-shape, or a Y-shape.

12. A micro actuator for a wearable drug delivery device, comprising:
a force transfer assembly including a magnet between a first focusing element and a second focusing element, wherein the second focusing element includes a yoke;
a first pair of drive coils; and
a second pair of drive coils,
wherein each drive coil of the first pair of drive coils is energized to a magnetic polarity that is opposite the other drive coil in order to attract or repel the first focusing element and the second focusing element in a first direction, and
each drive coil of the second pair of drive coils is energized to a magnetic polarity that is opposite the other drive coil in order to attract or repel the first focusing element and the second focusing in a second direction opposite the first direction.

13. A micro actuator of claim 12, comprising:
a main structure configured to hold the force transfer assembly, each drive coil of the first pair of drive coils, and each drive coil of the second pair of drive coils in alignment to enable rotation of the force transfer element.

14. The micro actuator of claim 13, wherein the main structure further comprises:
snap features configured to receive the spindle and operable to provide axial and radial bearing-like features for the spindle.

15. The micro actuator of claim 13, wherein the main structure further comprises:
a pocket to receive and hold each respective drive coil of the first pair of drive coils and each respective drive coil of the second pair of drive coils, and
wherein the pocket is configured to maintain alignment of each respective drive coil of the first pair of drive coils and each respective drive coil of the second pair of drive coils with a gap between the first focusing element and the second focusing element.

16. The micro actuator of claim 12, wherein each drive coil of the first pair of drive coils is wound from the same piece of wire, wherein the wire is wound in a first direction for a first respective drive coil of the first pair of drive coils and in a second and opposite direction for a second respective drive coil of the first pair of drive coils.

17. The micro actuator of claim 12, wherein the yoke comprises:
yoke extensions operable to interact with a drive mechanism of the wearable drug delivery device.

18. The micro actuator of claim 12, wherein the yoke comprises:
yoke extensions extending from either the first focusing element or the second focusing element in which the yoke has a T-shape, a U-shape, or a Y-shape.

19. The micro actuator of claim 12, further comprising:
a spindle,
wherein:
the magnet has an opening through which the spindle passes,
the first focusing element includes a first spindle opening and the second focusing element includes a second spindle opening, and
the spindle is configured to protrude through the first spindle opening and the second spindle opening.

20. The micro actuator of claim 19, wherein:
the spindle includes a keying structure configured to interlock with the first spindle opening and the second spindle opening.
